(19) United States
(12) Patent Application Publication
(10) Pub. No.: US 2007/0259973 A1
(43) Pub. Date: Nov. 8, 2007

(54) OPTIMIZED HYDROCARBON SYNTHESIS PROCESS

(75) Inventors: Kenneth Agee, Bixby, OK (US); Rafael Espinoza, Ponca City, OK (US)

Correspondence Address: BAKER & MCKENZIE LLP, Pennzoil Place, South Tower, 711 Louisiana, Suite 3400, HOUSTON, TX, 77002-2716, US

(73) Assignee: SYNTROLEUM CORPORATION, TULSA, OK (US)

(21) Appl. No.: 11/743,766

(22) Filed: May 3, 2007

Related U.S. Application Data
(60) Provisional application No. 60/746,302, filed on May 3, 2006.

(51) Int. Cl. C07C 27/06

(52) U.S. Cl. 518/702

(57) ABSTRACT

An optimized Fischer-Tropsch process utilizing a diluted synthesis and one or more Fischer-Tropsch reactors having an overall CO conversion of at least 90%.

### CROSS REFERENCE TO RELATED APPLICATIONS

This application claims priority to U.S. Provisional Application No. 60/746,302, filed May 3, 2006, which is incorporated herein in its entirety.

### FEDERALLY SPONSORED RESEARCH

Not applicable.

### REFERENCE TO MICROFICHE APPENDIX

Not applicable.

### FIELD OF THE INVENTION

This invention relates to a Fischer-Tropsch process wherein a nitrogen diluted synthesis gas is employed and more specifically to a Fischer-Tropsch process utilizing at least two Fischer-Tropsch reactors in series, with a carbon monoxide conversion in a single stage maintained at greater than 60% with an overall conversion of at least 90%.

### BACKGROUND OF THE INVENTION

Fischer-Tropsch processes for converting synthesis gas (“syngas”) into higher carbon number hydrocarbons are well known. The Fischer-Tropsch (“FT”) reaction for converting syngas, which is composed primarily of carbon monoxide (CO) and hydrogen gas (H₂), may be characterized by the following general reaction:

2nH₂+nCO→(—CH₂—)ₙ+nH₂O (1)

### Non-reactive components, such as nitrogen, may also be included or mixed with the syngas.

The syngas is delivered to a synthesis unit, which includes a Fischer-Tropsch reactor containing a Fischer-Tropsch catalyst. The hydrocarbon products of a Fischer-Tropsch synthesis generally include a wide range of carbon number, ranging from between about 1 and about 100. The end products which may be recovered from the Fischer-Tropsch synthesis product (“synthetic crude” or “syncrude”) following separation, hydroprocessing or other upgrading, include but are not limited to liquefied petroleum gas (“LPG”), naphtha, middle distillate fuels, e.g. jet and diesel fuels, and lubricant basestocks.

Fischer-Tropsch hydrocarbon synthesis catalysts have been studied widely by a number of researchers in recent years. Currently preferred processes include slurry bubble column processes wherein the Fischer-Tropsch catalysts typically comprise cobalt or ruthenium, cobalt and ruthenium or cobalt and a promoter. Currently used Fischer-Tropsch catalysts are typically supported on metal oxides such as alumina, silica, titanium, silica-alumina and the like.

Promoters can be used to enhance the activity of or the stability of cobalt or ruthenium catalysts. For example, ruthenium has been used to promote cobalt catalysts supported on either titania or alumina. Supported ruthenium catalysts have also been used for Fischer-Tropsch hydrocarbon. Ruthenium and zirconium have been used to promote cobalt supported on silica for use as Fischer-Tropsch catalysts.

Despite the fact that Fischer-Tropsch synthesis has been used at commercial scales since the early twentieth century, room for improvement exists, including in the areas of obtaining high conversion and dissipating heat generated in Fischer-Tropsch reactors. Since hydrocarbon synthesis is an exothermic reaction, heat must be removed from the reactor to avoid hot spots, catalyst deactivation, and loss of selectivity at higher temperatures. Lack of efficient heat removal can lead to much higher temperatures in the reactor which, while increasing carbon monoxide conversion, severely debits the selectivity to preferred higher hydrocarbons. At the same time, increasing conversion generates more heat and thus, a greater burden on heat exchange facilities. Thus the goals of high conversion and efficient heat transfer tend to oppose each other. To alleviate the problem, lower conversion in a first stage can be accommodated, thereby reducing the heat load in a first stage. However, this reduced conversion must be made up in a second or later stage in order to achieve sufficient conversion for commercial feasibility.

Many factors must be balanced in a commercial Fischer-Tropsch reactor such as, temperature, pressure, flow, conversion, H₂/CO ratio, etc. U.S. Pat. No. 4,169,120 teaches limiting the conversion for a single stage to a range of 40 to 60%. The reason given for limiting the conversion is to limit the build up of by-product water which can cause catalyst deactivation. If the conversion is limited to 60% per stage, however, two reactor stages only achieve an overall conversion of 84% and a third stage would be required to achieve an overall conversion in excess of 90%.

### SUMMARY OF THE INVENTION

According to the present invention, an optimized hydrocarbon synthesis process is provided and comprises reacting a first synthesis gas stream comprising hydrogen, carbon monoxide and from about 20 to about 60 volume percent diluent in a Fischer-Tropsch reactor in the presence of a catalyst comprising cobalt, ruthenium or cobalt and ruthenium supported on a support comprising at least one inorganic metal oxide selected from Group IIIA, IIIB, IVB, VB, VIB, and VIIB metal oxides alumina, silica, silica-alumina, and combinations thereof wherein at least one Fischer-Tropsch reactor has CO conversion of at least 60% and an overall CO conversion of at least 90%. Particularly preferred catalysts are catalysts comprising cobalt and ruthenium supported on alumina.

### BRIEF DESCRIPTION OF THE DRAWINGS

FIG. 1 is a schematic diagram of an embodiment of the process of the present invention.

### DESCRIPTION OF THE PREFERRED EMBODIMENTS

The term “Cₓ”, where x is a number greater than zero, refers to a hydrocarbon compound having predominantly a carbon number of x. As used herein, the term Cₓ, may be modified by reference to a particular species of hydrocarbons, such as, for example, C₅ olefins. In such instance, the term means an olefin stream comprised predominantly of pentenes but which may have impurity amounts, i.e. less than about 10%, of olefins having other carbon numbers such as hexene, heptene, propene, or butene. Similarly, the term “Cₓ+” refers to a stream wherein the hydrocarbons are predominantly those having a hydrocarbon number of x or greater but which may also contain impurity levels of hydrocarbons having a carbon number of less than x. For example, the term C₁₅₊ means hydrocarbons having a carbon number of 15 or greater but which may contain impurity levels of hydrocarbons having carbon numbers of less than 15.

The term “Cₓ—C_{y}”, where x and y are numbers greater than zero, refers to a mixture of hydrocarbon compounds wherein the predominant component hydrocarbons, collectively about 90% or greater by weight, have carbon numbers between x and y. For example, the term C₅-C₉ hydrocarbons means a mixture of hydrocarbon compounds which is predominantly comprised of hydrocarbons having carbon numbers between 5 and 9 but may also include impurity level quantities of hydrocarbons having other carbon numbers.

Unless otherwise specified, all quantities, percentages and ratios herein are by weight.

It has surprisingly been found that higher conversions can be sustained with no noticeable impact on the catalyst deactivation rate. The impact of the by product water is directly related to the partial pressure of the water. Embodiments of the invention utilize a synthesis gas that is diluted with a gas which is not reactive in the Fischer-Tropsch reaction. For example, synthesis gas generated with air or oxygen-enriched air has a substantial degree of nitrogen dilution, containing between about 20% to about 60% nitrogen. The dilution of the synthesis gas reduces the partial pressure of the water produced for a given conversion. Therefore it has now been shown that operation of one or more stages of Fischer-Tropsch reactions at CO conversions well above 60% per stage can be achieved and is desirable. According to embodiments of the present invention, the conversion of carbon monoxide in each Fischer-Tropsch reactor, i.e., each stage, exceeds 60%. In some embodiments of the invention, single stage conversion is up to 95%. As an example of the impact of higher single stage conversion rates on overall conversion, two FT reactors operating in series, each with 70% conversion results in overall conversion of carbon monoxide of 91%. Similarly, if each of two FT reactors has a 75% conversion rate, the overall CO conversion rate is 93.75%. Therefore, embodiments of the present invention permit the use of fewer stages resulting in substantial capital savings. As used herein the terms “% CO conversion” and “% conversion” refer to the percentage of CO in an initial feed stream which reacts by way of a Fischer-Tropsch reaction to form higher hydrocarbons on a once-through basis, i.e., achieved during a single pass through the Fischer-Tropsch reactor. The terms “overall CO conversion” and “overall conversion” mean the CO converted to higher hydrocarbons on a once-through basis after passing through all of the Fischer-Tropsch reactors in a given process. When referred to in connection with a specific Fischer-Tropsch reactor, the terms “CO conversion” and “conversion” mean the CO converted to higher hydrocarbons on a once-through basis upon passing solely through such specific Fischer-Tropsch reactor.

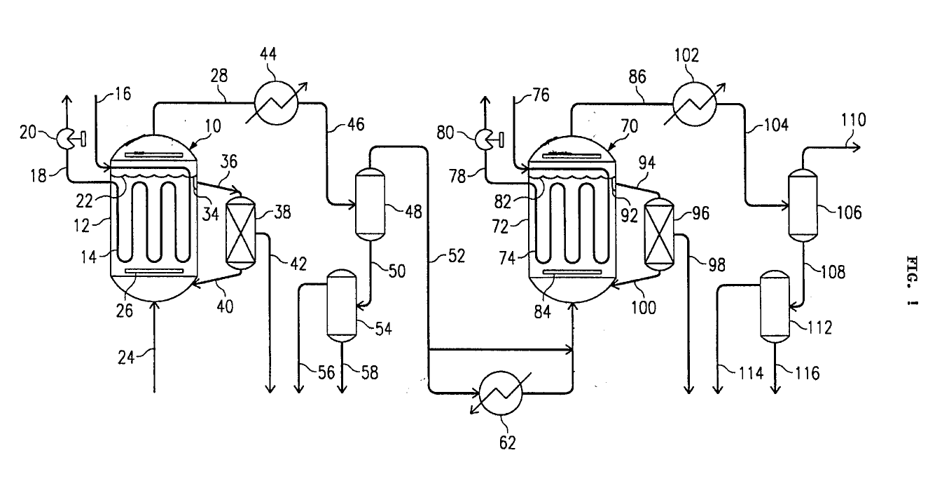

FIG. 1 will be discussed by reference to the use of a synthesis gas stream produced by the use of air or oxygen-enriched air (collectively or individually referred to as “air”) as the oxidant. However, synthesis gas produced using oxygen could be used in embodiments of the invention but in such embodiments, dilution with nitrogen or other non-reactive gas may be used to provide a produced water partial pressure within the limits described and claimed herein. FIG. 1 is a schematic diagram of an embodiment of the present invention including a first stage reactor 10 and a second stage reactor 70. First stage reactor 10 comprises a first vessel 12 which includes a plurality of heat exchange tubes 14 for the removal of heat. Coolant 16 is supplied to heat exchange tubes 14 and recovered as recovery stream 18. A back pressure control valve 20 enables the control of the pressure thereby regulating the temperature in first vessel 12. In a preferred embodiment, the coolant 16 is water and the recovered coolant stream 18 is steam. In some embodiments, the recovered coolant stream 18 is re-cooled or otherwise appropriately disposed or recycled. As described in connection with FIG. 1, first vessel 12 is a slurry bubble column reactor which contains a slurry comprised primarily of Fischer-Tropsch reaction products in which Fischer-Tropsch catalyst is suspended. Fischer-Tropsch particle size is typically less than about 150 micron in diameter. The Fischer-Tropsch catalyst particles are fluidized in the liquid by a synthesis gas 24 passed into first vessel 12. The synthesis gas 24 is dispersed as a series of small bubbles for movement upwardly through first vessel 12 through a gas distributor 26 or other appropriate means. The slurry level is preferably maintained at a level 22. An overhead gaseous stream 28 is recovered.

A Fischer-Tropsch liquid product is recovered from first vessel 12 by positioning a weir 34 in first vessel 12 below level 22 so that Fischer-Tropsch liquid product can collect in weir 34 and de-gas, thereby increasing the density of Fischer-Tropsch liquid product. The denser Fischer-Tropsch liquid product 36 is passed through a filter 38 from which a first Fischer-Tropsch liquid product 42 comprised primarily of C₁₇₊ hydrocarbon liquids is recovered. Following removal of the first Fischer-Tropsch liquid product 42, a remaining slurry 40 is returned via to a lower portion of first vessel 12. First Fischer-Tropsch liquid product 42 is removed at a rate sufficient to maintain the liquid level 22 in first vessel 12 at a desired level. Level 22 is maintained such that a sufficient disengaging zone is maintained above the slurry. Under preferred operating conditions, all liquid hydrocarbon recovery 42 from first vessel 12 is through filter 38.

The synthesis gas 24 charged to first vessel 12 in the embodiment shown in FIG. 1 is typically produced by an autothermal reactor or the like. In some processes oxygen is used as the primary oxidant. In such instances, the synthesis gas stream 24 will contain hydrogen, carbon monoxide, carbon dioxide, and water unless the water has been removed prior to charging the synthesis gas stream to first vessel 12. Alternatively, the synthesis gas 24 may be produced by using air or oxygen-enriched air as the oxidant gas stream. In such instances, the synthesis gas stream 24 will contain not insignificant quantities, i.e., from about 20 to about 60 percent nitrogen, at the point such syngas is fed into first vessel 12. Water may be removed from the synthesis gas produced by the use of air or oxygen-enriched air as the oxidant gas prior to being fed into first vessel 12.

The gaseous stream 28 comprises gaseous hydrocarbons, hydrogen, carbon monoxide, and nitrogen. The gaseous stream 28 is cooled to a temperature below about 150° F. by any acceptable processing means. For example, gaseous stream 28 may be passed to a heat exchanger 44. Preferably the temperature of gaseous stream 28 is reduced to a temperature of about less than about 100° F. Cooled gaseous stream 46 is then passed to a first separator 48 from which a second synthesis gas stream 52 is recovered. A recovered liquid stream 50 is recovered from first separator 48 and passed to a second separator 54 where the recovered liquid stream 50 is separated into a first recovered hydrocarbon stream 56 and an aqueous stream 58. First recovered hydrocarbon stream 56 is comprised primarily of C₅-C₁₇ hydrocarbons. Aqueous stream 58 is comprised primarily of water. The second synthesis gas stream 52 may be heated to a temperature suitable for charging to second stage reactor 70 using any acceptable processing means. For example, second synthesis gas stream 52 may be passed to a heat exchanger 62. For injection into second stage reactor 70, second synthesis gas stream 52 is typically heated to a temperature between about 300° and about 400° F. In some embodiments of the invention, second synthesis gas stream 52 may be passed from first separator 48 directly to second stage reactor 70 without additional heating.

Second stage reactor 70 comprises a vessel 72 including a plurality of heat exchange tubes 74 which are supplied with coolant 76 with recovered coolant 78 being recovered. In a preferred embodiment, the coolant 76 is water and the recovery stream 78 is steam. In some embodiments, the recovery stream 78 is re-cooled or otherwise appropriately disposed or recycled. A back pressure control valve 80 controls the pressure in heat exchange tubes 74 thereby regulating the temperature in second vessel 72. Second stage reactor 70 is a slurry bubble column reactor which contains a slurry comprised primarily of Fischer-Tropsch reaction products in which Fisher-Tropsch catalyst is suspended. A gas distributor 84 is positioned in the lower part of second vessel 72 to disperse second synthesis gas 52 into the slurry for movement upward through second vessel 72 as finely dispersed bubbles. A second gaseous stream 86 is recovered overhead.

A weir 92 is used to collect a portion of the slurry for withdrawal. The withdrawn slurry 94 is degassed before being passed through a second filter 96 from which a second Fischer-Tropsch liquid product 98 comprised primarily of C₁₇₊ hydrocarbons is removed. Following removal of the second Fischer-Tropsch liquid product 98 from withdrawn slurry 94, the remaining slurry 100 is reinjected into second vessel 72.

The second gaseous stream 86 which contains primarily gaseous hydrocarbons, hydrogen, carbon monoxide, and nitrogen, is recovered. Second gaseous stream 86 may be cooled to a temperature between about 100° F. and about 150° F. and preferably to a temperature of about 100° F. using any acceptable processing means. For example, as shown in FIG. 1, second gaseous stream 86 may be passed to a heat exchanger 102. Cooled gaseous stream 104 is then passed to a separator 106. From separator 106 a third synthesis gas 110 and a second liquid stream 108 are recovered. The liquid stream 108 is passed to a second liquid separator 112 where a second recovered hydrocarbon liquid stream 114 comprised primarily of C₅-C₁₇ hydrocarbons is separated from an aqueous stream 116 which is comprised primarily of water.

The third synthesis gas stream 110 may be passed for further reaction in an additional Fischer-Tropsch reactor. Alternatively, the third synthesis gas stream 110 may be further processed by adsorption, absorption, or low temperature processes to recover light hydrocarbons with any remaining gaseous components useful as low BTU fuel gas.

The present process is particularly adapted to the use of a synthesis gas containing nitrogen. Such synthesis gas streams are not well adapted to recycle, for increasing the conversion rate of other gases. Minimal recycling may be employed, if at all. For example, CO₂ could be removed and recycled to synthesis gas generation or light olefins could be extracted and recycled to FT synthesis but recycle of nitrogen containing synthesis gas should be avoided.

The catalyst used in the present invention comprises cobalt, ruthenium, or cobalt and ruthenium supported on a support comprising an inorganic metal oxide selected from Group IIIA, IIIB, IVB, VB, VIB and VIIIB metal oxides, alumina, silica, silica-alumina and combinations thereof. The catalyst used in first stage reactor 10 and in second stage reactor 70 may be the same or different within the parameters set forth herein for the catalyst. Preferably the catalyst support comprises primarily alumina, titania, silica, silica-alumina, and combinations thereof with the preferred support comprising alumina.

Further, the catalyst may include a promoter. The promoter may be selected from those known to those skilled in the art for use with supported cobalt, ruthenium, or cobalt and ruthenium catalysts. Suitable promoters are selected from a group consisting of zirconium, titanium, thenium, cerium, hafnium, ruthenium, and uranium. In a preferred embodiment of the invention, the Fischer-Tropsch catalyst is cobalt supported on alumina promoted with ruthenium.

The liquid hydrocarbon products recovered from the process may be processed together or separately. First and second recovered hydrocarbon streams 56 and 114 are comprised primarily of C₅-C₁₇ hydrocarbons. First and second Fischer-Tropsch liquid products 42 and 98 are comprised primarily of C₁₇₊ hydrocarbons. Each of the streams 42, 98, 56, and 114 may be further processed for use as a variety of fuels, as chemical feedstocks and the like as is known to those skilled in the art.

Having thus described the present invention by reference to certain of its preferred embodiments, it is noted that the embodiments described are illustrative rather than limiting in nature and many variations and modifications are possible within the scope of the present invention. For example, while FIG. 1 illustrates a process using synthesis gas produced using air or oxygen-enriched air, synthesis gas produced with pure oxygen may also be used in embodiments of the invention. Similarly, commonly known reactor types and configurations, such as fixed bed reactors, heating and cooling means, such as shell tube heat exchangers, and heat recovery systems may be used in embodiments of the invention.

We claim:

1. A process to produce hydrocarbons comprising the steps of:
passing a diluted synthesis gas through one or more Fischer-Tropsch reactors in series wherein the CO conversion in at least one of the Fischer-Tropsch reactors is greater than 60% and the overall CO conversion is at least 90%.

2. The process of claim 1, wherein the synthesis gas is generated by partial oxidation or auto thermal reforming of light hydrocarbons with air or oxygen-enriched air.

3. The process of claim 1, wherein the synthesis gas is generated by gasifying coal, petroleum coke, residual hydrocarbons, or biomass with air or oxygen-enriched air.

4. The process of claim 1, wherein the synthesis gas stream is scrubbed to remove trace contaminants such as NH₃ and HCN to a level below about 300 ppb.

5. The process of claim 1, wherein the synthesis gas is generated at a pressure sufficient to flow directly to the FT reactor(s) without further need of compression.

6. The process of claim 1, wherein the synthesis gas is generated at a pressure that is lower than a first Fischer-Tropsch reactor(s) and is therefore compressed to a pressure from 200 psig to 600 psig and fed to the first stage FT reactor.

7. The process of claim 1, wherein a heavy hydrocarbon product is removed through internal filters located in a slurry zone.

8. The process of claim 1, wherein a heavy hydrocarbon product is removed through external filters.

9. The process of claim 1, wherein an overhead stream is cooled to an intermediate temperature to remove a portion of a condensable products as a liquid.

10. The process of claim 1, wherein a product is recovered from a tail gas of a final reactor stage by a cryogenic process, absorption, or adsorption.

11. The process of claim 1, wherein the CO conversion in at least one Fischer-Tropsch reactor is greater than 60% and less than about 80%.

12. The process of claim 1, wherein the CO conversion in at least one Fischer-Tropsch reactor is between about 70% and about 80%.

13. The process of claim 1, wherein the CO conversion in at least one Fischer-Tropsch reactor is between about 72% and about 76%.

14. The process of claim 1, wherein the overall CO conversion is at least 92%.

15. The process of claim 1, wherein the overall CO conversion is at least 94%.

16. The process of claim 1, wherein the partial pressure of water in each Fischer-Tropsch reactor(s) is less than 60 bar.

17. The process of claim 1, wherein the partial pressure of water in each Fischer-Tropsch reactor(s) is between about 40 bar and about 60 bar.

18. The process of claim 1, wherein the partial pressure of water in each Fischer-Tropsch reactor(s) is between about 50 bar and about 60 bar.

19. The process of claim 1, wherein the partial pressure of water in each Fischer-Tropsch reactor(s) is about 55 bar.

20. The process of claim 1, wherein the diluted synthesis gas comprises about 30% nitrogen by volume.

21. The process of claim 1, wherein the diluted synthesis gas contains between about 20% and about 60% nitrogen by volume.

22. The process of claim 1, wherein the Fischer-Tropsch reactor(s) contains a Fischer-Tropsch reaction catalyst comprising cobalt supported on alumina.

23. The process of claim 1, wherein the Fischer-Tropsch reactor(s) contains a Fischer-Tropsch reaction catalyst comprising cobalt and ruthenium supported on alumina.

24. The process of claim 1, wherein the Fischer-Tropsch reactor(s) is operated at temperatures of between about 380 and about 500° F.

25. The process of claim 1, wherein the Fischer-Tropsch reactor(s) is operated at pressures of between about 15 and about 40 atmospheres.

26. The process of claim 1, wherein the synthesis gas is diluted with one or more gasses selected from the group consisting of nitrogen, carbon dioxide, and methane.

27. The process of claim 1, wherein at least one of the Fischer-Tropsch reactors has a CO conversion of at least 90%.
